(19) United States
(12) Patent Application Publication
(10) Pub. No.: US 2007/0172958 A1
(43) Pub. Date: Jul. 26, 2007

(54) Methods for detecting 14-hydroxy codeinone and codeinone

(75) Inventors: Bassam Freiha, Warwick, RI (US); John Decubellis, Lincoln, RI (US)

Correspondence Address: DAVIDSON, DAVIDSON & KAPPEL, LLC, 485 SEVENTH AVENUE, 14TH FLOOR, NEW YORK, NY, 10018, US

(73) Assignee: EURO-CELTIQUE S.A., Luxembourg (LU)

(21) Appl. No.: 10/588,872

(22) PCT Filed: Mar. 30, 2005
(86) PCT No.: PCT/US05/10665

§ 371 Date: Aug. 10, 2006

Related U.S. Application Data
(60) Provisional application No. 60/557,502, filed on Mar. 30, 2004.
(60) Provisional application No. 60/648,629, filed on Jan. 31, 2005.

(51) Int. Cl. G01N 33/00

(52) U.S. Cl. 436/93

(57) ABSTRACT

Disclosed is a method of detecting the presence of 14-hydroxycodeinone and/or codeinone in an oxycodone preparation.

### CROSS REFERENCE TO RELATED APPLICATIONS

This application claims priority from and incorporates herein by reference U.S. Patent Application No. 60/557,502, filed Mar. 30, 2004, and U.S. Patent Application No. 60/648,629, filed Jan. 31, 2005.

### FIELD OF THE INVENTION

An improved method is disclosed for detecting the presence of 14-hydroxycodeinone and/or codeinone in an oxycodone preparation.

### BACKGROUND OF THE INVENTION

There is a continuing need for improved methods to determine the presence and amount of impurities in pharmaceutical preparations.

In the pharmaceutical industry, pharmaceutical compounds must be inspected to determine the presence of undesirable impurities. To be detected, the amount of impurity must usually be present above a certain threshold level which depends upon the assay technique utilized. For example, if a particular assay has a limit of quantitation of 100 ppm for a particular impurity in a sample, that assay would generally be unable to determine if the impurity were present in any amount less than 100 ppm. Such limitations can be problematic in assaying pharmaceutical preparations for the presence of impurities.

Oxycodone hydrochloride (commercialized as Oxycontin®, OxyIR®, and OxyFast®) is a narcotic analgesic widely prescribed for the treatment of pain (Physicians Desk Reference, 56^{th} Ed., p. 2912-2918 (2002); Merck Index, 12^{th} Ed., 7093).

Commonly assigned U.S. Provisional Application entitled “Process For Preparing Oxycodone Hydrochloride Having Less Than 25 PPM 14-Hydroxycodeinone and Compositions Thereof” filed Mar. 30, 2004 relates to a process for reducing the amount of 14-hydroxycodeinone in an oxycodone hydrochloride preparation, and compositions having reduced amounts of 14-hydroxycodeinone.

There exists a need in the art to provide an improved method for detecting impurities in an oxycodone preparation. More specifically, there exists a need in the art to provide more sensitive methods for detecting 14-hydroxycodeinone and codeinone in oxycodone preparations.

### OBJECTS AND SUMMARY OF THE INVENTION

It is an object of the present invention to provide an improved method for detecting impurities in an oxycodone preparation.

It is an object of certain embodiments of the present invention to provide an improved method for detecting 14-hydroxycodeinone in an oxycodone preparation.

It is an object of certain embodiments of the present invention to provide an improved method for detecting codeinone in an oxycodone preparation.

It is a further object of certain embodiments of the present invention to provide a method for detecting the presence of impurities in an oxycodone preparation, which method exhibits increased sensitivity.

It is a further object of certain embodiments of the present invention to provide a method for detecting 14-hydroxycodeinone in an oxycodone preparation when the 14-hydroxycodeinone is present in the preparation in an amount of less than 250 ppm.

It is a further object of certain embodiments of the present invention to provide a method for detecting codeinone in an oxycodone preparation when the codeinone is present in the preparation in an amount of less than 250 ppm.

In accordance with the above objects, the present invention provides, in part, a method of determining the amount of 14-hydroxycodeinone contained in an oxycodone preparation utilizing two standard solutions and one sample solution, the method comprising:
(a) preparing for analysis by a detection system (e.g., an HPLC system) a standard solution comprising 14-hydroxycodeinone in a known concentration;
(b) preparing for analysis by the detection system a second standard solution comprising 14-hydroxycodeinone in a second known concentration;
(c) preparing for analysis by the detection system a sample solution comprising oxycodone from the oxycodone preparation;
(d) analyzing the first standard solution using the detection system to obtain a measurable quantification (e.g., a measurable peak area) of 14-hydroxycodeinone in the first known concentration;
(e) analyzing the second standard solution using the detection system to obtain a measurable quantification of 14-hydroxycodeinone in the second known concentration;
(f) analyzing the sample solution of step (c) using the detection system to obtain a measurable quantification of 14-hydroxycodeinone in the sample solution; and
(g) determining the amount of 14-hydroxycodeinone in the oxycodone preparation based on a comparison of the quantifications obtained for the first and second standard solution with the quantification obtained for the sample solution.

In certain embodiments, the present invention is further directed to a method of determining the amount of 14-hydroxycodeinone contained in an oxycodone preparation utilizing two standard solutions and one sample solution, the method comprising:
(a) preparing for analysis by a detection system a first standard solution comprising 14-hydroxycodeinone in a first known concentration;
(b) preparing for analysis by the detection system a second standard solution comprising 14-hydroxycodeinone in a second known concentration;
(c) preparing for analysis by the detection system a sample solution of oxycodone from the oxycodone preparation, wherein the concentration of oxycodone in the sample solution is from about 10 mg/mL to the solubility limit of the oxycodone, or from about 50 mg/mL to about 150 mg/mL;
(d) analyzing the first standard solution using the detection system to obtain a measurable quantification of 14-hydroxycodeinone in the known concentration;
(e) analyzing the second standard solution using the detection system to obtain a measurable quantification of 14-hydroxycodeinone in the second known concentration;
(f) analyzing the sample solution of step (b) using the detection system to obtain a measurable quantification of 14-hydroxycodeinone in the sample solution; and
(g) determining the amount of 14-hydroxycodeinone in the oxycodone preparation based on a comparison of the quantifications obtained for the first and second standard solution with the quantification obtained for the sample solution (e.g., by using the calculations of Example 2 or an equivalent mathematical formula).

In accordance with the above objects, the present invention provides, in part, a method of determining the amount of codeinone contained in an oxycodone preparation utilizing two standard solutions and one sample solution, the method comprising:
(a) preparing for analysis by a detection system (e.g., an HPLC system) a standard solution comprising codeinone in a known concentration;
(b) preparing for analysis by the detection system a second standard solution comprising codeinone in a second known concentration;
(c) preparing for analysis by the detection system a sample solution comprising oxycodone from the oxycodone preparation;
(d) analyzing the first standard solution using the detection system to obtain a measurable quantification (e.g., a measurable peak area) of codeinone in the first known concentration;
(e) analyzing the second standard solution using the detection system to obtain a measurable quantification of codeinone in the second known concentration;
(f) analyzing the sample solution of step (c) using the detection system to obtain a measurable quantification of codeinone in the sample solution; and
(g) determining the amount of codeinone in the oxycodone preparation based on a comparison of the quantifications obtained for the first and second standard solution with the quantification obtained for the sample solution.

In certain embodiments, the present invention is further directed to a method of determining the amount of codeinone contained in an oxycodone preparation utilizing two standard solutions and one sample solution, the method comprising:
(a) preparing for analysis by a detection system a first standard solution comprising codeinone in a first known concentration;
(b) preparing for analysis by the detection system a second standard solution comprising codeinone in a second known concentration;
(c) preparing for analysis by the detection system a sample solution of oxycodone from the oxycodone preparation, wherein the concentration of oxycodone in the sample solution is from about 10 mg/mL to the solubility limit of the oxycodone, or from about 50 mg/mL to about 150 mg/mL;
(d) analyzing the first standard solution using the detection system to obtain a measurable quantification of codeinone in the known concentration;
(e) analyzing the second standard solution using the detection system to obtain a measurable quantification of codeinone in the second known concentration;
(f) analyzing the sample solution of step (b) using the detection system to obtain a measurable quantification of codeinone in the sample solution; and
(g) determining the amount of codeinone in the oxycodone preparation based on a comparison of the quantifications obtained for the first and second standard solution with the quantification obtained for the sample solution (e.g., by using the calculations of Example 2 or an equivalent mathematical formula).

In accordance with the above objects, the present invention provides, in part, a method of determining the amount of 14-hydroxycodeinone contained in an oxycodone preparation utilizing one standard solution and one sample solution, the method comprising:
(a) preparing for analysis by a detection system (e.g., an BPLC system) a standard solution comprising 14-hydroxycodeinone in a known concentration;
(b) preparing for analysis by the detection system a sample solution comprising oxycodone from the oxycodone preparation;
(c) analyzing the standard solution using the detection system to obtain a measurable quantification (e.g., a measurable peak area) of 14-hydroxycodeinone in the known concentration;
(d) analyzing the sample solution of step (b) using the detection system to obtain a measurable quantification of 14-hydroxycodeinone in the sample solution; and
(e) determining the amount of 14-hydroxycodeinone in the oxycodone preparation based on a comparison of the quantifications obtained for the standard solution with the quantification obtained for the sample solution.

In certain embodiments, the present invention is further directed to a method of determining the amount of 14-hydroxycodeinone contained in an oxycodone preparation utilizing one standard solution and one sample solution, the method comprising:
(a) preparing for analysis by a detection system a standard solution comprising 14-hydroxycodeinone in a known concentration;
(b) preparing for analysis by the detection system a sample solution of oxycodone from the oxycodone preparation, wherein the concentration of oxycodone in the sample solution is from about 10 mg/mL to the solubility limit of the oxycodone, or from about 50 mg/mL to about 150 mg/mL;
(c) analyzing the standard solution using the detection system to obtain a measurable quantification of 14-hydroxycodeinone in the known concentration;
(d) analyzing the sample solution of step (b) using the detection system to obtain a measurable quantification of 14-hydroxycodeinone in the sample solution; and
(e) determining the amount of 14-hydroxycodeinone in the oxycodone preparation based on a comparison of the quantifications obtained for the standard solution with the quantification obtained for the sample solution (e.g., by using the calculations of Example 2 or an equivalent mathematical formula).

In accordance with the above objects, the present invention provides, in part, a method of determining the amount of codeinone contained in an oxycodone preparation utilizing one standard solution and one sample solution, the method comprising:
(a) preparing for analysis by a detection system (e.g., an HPLC system) a standard solution comprising codeinone in a known concentration;
(b) preparing for analysis by the detection system a sample solution comprising oxycodone from the oxycodone preparation;
(c) analyzing the standard solution using the detection system to obtain a measurable quantification (e.g., a measurable peak area) of codeinone in the known concentration;
(d) analyzing the sample solution of step (b) using the detection system to obtain a measurable quantification of codeinone in the sample solution; and
(e) determining the amount of codeinone in the oxycodone preparation based on a comparison of the quantifications obtained for the standard solution with the quantification obtained for the sample solution.

In certain embodiments, the present invention is further directed to a method of determining the amount of codeinone contained in an oxycodone preparation utilizing one standard solution and one sample solution, the method comprising:
(a) preparing for analysis by a detection system a standard solution comprising codeinone in a known concentration;
(b) preparing for analysis by the detection system a sample solution of oxycodone from the oxycodone preparation, wherein the concentration of oxycodone in the sample solution is from about 10 mg/mL to the solubility limit of the oxycodone, or from about 50 mg/mL to about 150 mg/mL;
(c) analyzing the standard solution using the detection system to obtain a measurable quantification of codeinone in the known concentration;
(d) analyzing the sample solution of step (b) using the detection system to obtain a measurable quantification of 14-hydroxycodeinone in the sample solution; and
(e) determining the amount of codeinone in the oxycodone preparation based on a comparison of the quantifications obtained for the standard solution with the quantification obtained for the sample solution (e.g., by using the calculations of Example 2 or an equivalent mathematical formula).

In conducting methods of the present invention utilizing two standard solutions, one of the first or second concentrations is greater than the other concentration. It is not necessary for the concentrations of the standard solutions to bracket the concentration of the sample solution.

In conducting methods of the present invention utilizing two standard solutions, the detection system used can be any appropriate detection system that can provide a measurable quantification (e.g., a measurable peak area) of the amount of 14-hydroxycodeinone and/or codeinone in the first standard solution, the amount of 14-hydroxycodeinone and/or codeinone in the second standard solution, and the amount of 14-hydroxycodeinone and/or codeinone in the sample solution. In certain embodiments, the detection system can be a liquid or gas chromatography system such as, e.g., a high performance liquid chromatography (“HPLC”) system. In certain embodiments, the step of determining the amount of 14-hydroxycodeinone in the oxycodone preparation is based on a linear calibration curve analysis of the first standard solution, the second standard solution and the sample solution utilizing an HPLC system. In certain embodiments, the linear calibration curve utilizes the peaks of the first and second solutions and point “0” on the curve to quantify the sample.

In conducting methods of the present invention utilizing one standard solution, the detection system used can be any appropriate detection system that can provide a measurable quantification (e.g., a measurable peak area) of the amount of 14-hydroxycodeinone and/or codeinone in the standard solution and the amount of 14-hydroxycodeinone and/or codeinone in the sample solution. In certain embodiments, the detection system can be a liquid or gas chromatography system such as, e.g., a high performance liquid chromatography (“HPLC”) system.

In certain embodiments, the HPLC system used according to a method of the present invention has a column that is maintained at a temperature from ambient temperature to about 60 degrees C., from about 30 degrees C. to about 60 degrees C., from about 40 degrees C. to about 60 degrees C., or about 40 degrees C., so as to obtain a measurable peak area of 14-hydroxycodeinone and/or codeinone.

In the embodiments of the present invention, the detection systems utilized to analyze the standard solution(s) and the sample solution can be the same system, or different systems. For example, the same system can be used sequentially for all of the analysis, or separate systems can be utilized wherein the results are capable of comparison, either directly or by computation.

In an alternate embodiment, the invention is directed to a method of determining the amount of 14-hydroxycodeinone contained in an oxycodone preparation utilizing no standard and two sample solutions, comprising:
(a) preparing for analysis by a detection system a first sample solution from the oxycodone preparation; wherein the concentration of oxycodone in the first sample solution is sufficient to quantify the 14-hydroxycodeinone contained therein;
(b) preparing for analysis by the detection system a second sample solution from the oxycodone preparation, wherein the concentration of oxycodone in the second sample solution is sufficient to quantify the oxycodone contained therein, and wherein the second sample solution has a concentration of oxycodone that is less than the concentration of oxycodone in the first sample solution;
(c) analyzing the first sample solution using a detection system to obtain a measurable peak area of 14-hydroxycodeinone;
(d) analyzing the second sample solution using the detection system to obtain a measurable peak area of oxycodone; and
(e) determining the amount of 14-hydroxycodeinone present in the oxycodone preparation based on the analysis of the first and second sample solutions.

In an alternate embodiment, the invention is directed to a method of determining the amount of codeinone contained in an oxycodone preparation utilizing no standard and two sample solutions, comprising:
(a) preparing for analysis by a detection system a first sample solution from the oxycodone preparation; wherein the concentration of oxycodone in the first sample solution is sufficient to quantify the codeinone contained therein;
(b) preparing for analysis by the detection system a second sample solution from the oxycodone preparation, wherein the concentration of oxycodone in the second sample solution is sufficient to quantify the oxycodone contained therein, and wherein the second sample solution has a concentration of oxycodone that is less than the concentration of oxycodone in the first sample solution;
(c) analyzing the first sample solution using a detection system to obtain a measurable peak area of codeinone;
(d) analyzing the second sample solution using the detection system to obtain a measurable peak area of oxycodone; and
(e) determining the amount of codeinone present in the oxycodone preparation based on the analysis of the first and second sample solutions.

In these alternate embodiments utilizing no standard and two sample solutions, the oxycodone in the first sample solution can be at a concentration, e.g., from about 10 mg/mL to the solubility limit of the oxycodone, or from about 50 mg/mL to about 150 mg/mL; the oxycodone in the second sample solution can be at a concentration, e.g., from about 0.01 mg/mL to about 5 mg/mL; and/or the determination of the amount of 14-hydroxycodeinone or codeinone in the oxycodone preparation can be obtained by dividing the peak area of 14-hydroxycodeinone or codeinone in the first sample by [the peak area of oxycodone in the second sample solution multiplied by (the concentration of oxycodone in the first sample solution divided by the concentration of oxycodone in the second sample solution) multiplied by the 14-hydroxycodeinone or codeinone RRF (relative response factor) multiplied by the dilution factor] to obtain a quotient.

In certain embodiments, the oxycodone preparation is a pharmaceutically acceptable salt of oxycodone. Pharmaceutically acceptable salts of oxycodone include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, sodium dodecyl sulphate, and the like; amino acid salts such as arginate, asparginate, glutamate and the like. The pharmaceutically acceptable salts can include anhydrous forms and hydrous forms, e.g., monohydrates and dihydrates.

The oxycodone preparation can be oxycodone base or a pharmaceutically acceptable salt (e.g., an oxycodone active pharmaceutical ingredient (API), such as oxycodone hydrochloride U.S.P.) uncombined or combined with one or more other ingredients. For example, the oxycodone preparation can be a final pharmaceutical dosage form, or an intermediate preparation for a final dosage form, that can be tested for the presence of 14-hydroxycodeinone and/or codeinone, e.g., for quality assurance purposes. Preferably, the oxycodone preparation is oxycodone API and contains at least 95% oxycodone, at least 98% oxycodone, at least 99% oxycodone, or at least 99.9% oxycodone.

The term “ppm” as used herein means “parts per million”. For example, as used to refer to 14-hydroxycodeinone, “ppm” means parts per million of 14-hydroxycodeinone in the particular composition or sample thereof, e.g., oxycodone hydrochloride U.S.P.

For purposes of the present invention, the term “relative response factor” or “RRF” is a correction factor applied in chromatography systems, e.g., HPLC systems, to correct for the difference in response of different compounds, e.g., impurities, to the response of the reference standard.

### BRIEF DESCRIPTION OF THE DRAWINGS

FIG. 1 depicts a typical separation of the system suitability testing solution of Example 4.

FIG. 2 is an HPLC chromatogram for the working 100 ppm 14-hydroxycodeinone standard solution of Example 4.

FIG. 3 is an HPLC chromatogram of the sample of oxycodone active pharmaceutical ingredient of Example 4.

FIG. 4 is a representative HPLC chromatogram of the RTM solution of Example 6.

FIG. 5 is a representative HPLC chromatogram of the unspiked sample solution of Example 6.

FIG. 6 is a representative HPLC chromatogram of the 10 ppm working standard III solutions of Example 6.

### DETAILED DESCRIPTION OF THE INVENTION

The present invention provides an improved method whereby the presence of 14-hydroxycodeinone and/or codeinone in an amount less than 25 ppm can be determined.

In certain embodiments, this method utilizes HPLC technology and involves (i) taking a first known concentration of 14-hydroxycodeinone and/or codeinone in a first standard solution(s) and a second known concentration of 14-hydroxycodeinone and/or codeinone in a second standard solution(s) and determining the peak areas of 14-hydroxycodeinone and/or codeinone of the standard solutions; (ii) taking an oxycodone preparation having a 14-hydroxycodeinone and/or codeinone level in a concentration of less than 100 ppm and preparing a sample solution with a high concentration of oxycodone in order to increase the peak concentration for the 14-hydroxycodeinone and/or codeinone in the sample solution(s); and (iii) performing a linear calibration curve analysis of the two standards with the sample to determine the amount of 14-hydroxycodeinone and/or codeinone in the sample(s). Certain adjustments in chromatographic conditions can also be implemented to “sharpen” the 14-hydroxycodeinone peak.

In certain embodiments, this method utilizes HPLC technology and involves (i) taking a known concentration of 14-hydroxycodeinone and/or codeinone in a standard solution(s) and determining the peak areas of 14-hydroxycodeinone and/or codeinone of the standard solution(s); (ii) taking an oxycodone preparation and preparing a sample solution; and (iii) performing an analysis of the standard with the sample to determine the amount of 14-hydroxycodeinone and/or codeinone in the sample. Certain adjustments in chromatographic conditions can also be implemented to “sharpen” the 14-hydroxycodeinone and/or codeinone peak.

In the alternate embodiments wherein no standard and two sample solutions are used, the method of the present invention further involves increasing the concentration of oxycodone in the first sample solution in order to increase the peak area of 14-hydroxycodeinone to a level that can be accurately determined.

In certain embodiments, wherein no standard and two sample solutions are used, the increased concentration of the oxycodone in the first sample solution results in an increase in the peak area of oxycodone beyond the upper limit of the detector that can be accurately measured. Accordingly, a second sample solution is prepared at a lower concentration of oxycodone so as to produce a chromatographic peak for oxycodone that is on-scale and can be accurately quantified. This second solution can be prepared by diluting a portion of the first sample solution, by initially preparing a less concentrated solution, or by any other method, so long as the difference in concentrations between the first and second sample solutions is known.

In certain embodiments, wherein no standard and two sample solutions are used, the peak area of 14-hydroxycodeinone and/or codeinone from the high concentration first sample solution, and the peak area of oxycodone from the lower concentration second sample solution can be used to calculate the amount (e.g., percentage) of 14-hydroxycodeinone and/or codeinone in the original oxycodone preparation. An example of making such a calculation is: dividing the peak area of 14-hydroxycodeinone and/or codeinone by [the peak area of oxycodone multiplied by (the concentration of oxycodone in the first sample solution divided by the concentration of oxycodone in the second sample solution) multiplied by the 14-hydroxycodeinone and/or codeinone RRF multiplied by the dilution factor] to obtain a quotient. The quotient can then be optionally multiplied by 100 to obtain a percentage.

The methods of the present invention utilizing two standard solutions have a limit of quantitation of less than about 5 ppm. Accordingly, the methods of the present invention can provide for the detection of 14-hydroxycodeinone or codeinone levels in an oxycodone preparation where the 14-hydroxycodeinone or codeinone is present in an amount of less than 25 ppm, less than 20 ppm, less than 15 ppm, less than 10 ppm, about 5 ppm, or less than 5 ppm.

The methods of the present invention utilizing one standard solution can provide for the detection of 14-hydroxycodeinone or codeinone levels in an oxycodone preparation where the 14-hydroxycodeinone or codeinone is present in an amount of less than 25 ppm, less than 20 ppm, less than 15 ppm, less than 10 ppm, or less than 5 ppm.

Where the detection system is an HPLC system, the HPLC system typically includes at least the following components: an HPLC column packed with a suitable stationary phase; a mobile phase; a pump for directing the mobile phase through the column; and an HPLC compatible detector for detecting the presence of compounds eluting from the column.

Methods and apparatus for carrying out BPLC separations are described, for example, in J. Chromatography, 192:222-227 (1980); J. Liquid Chromatography 4:661-680 (1981); and J. Chromatography, 249:193-198 (1982), among many other publications.

In certain embodiments, the HPLC system is an adsorption chromatography system, an ion-exchange chromatography system, a size exclusion chromatography system, or the like. In one embodiment, the HPLC system is an adsorption chromatography system such as, for example, a normal phase chromatography system or a reverse phase chromatography system. In a more preferred embodiment, the HPLC system is a reverse phase chromatography system.

Reverse phase chromatography involves contacting a solution of a desired compound with a solid, hydrophobic support, or stationary phase, under conditions whereby the compound is adsorbed to the support. The compound is then eluted after washing by rinsing the support with an apolar organic solvent (i.e., the mobile phase). The stationary phase can comprise a support such as a silica-based support, to which are bonded various non-polar organic groups. Such bonded phases may be prepared, for example, by reacting surface silanol groups on the silica with an organo-chlorosilane, as known in the art. Silica-based supports include, for example, spherical silica particles, irregular silica particles or particulate substrates coated with silica. The particle size and porosity should be appropriately selected for separation of the specific components in the assay.

The mobile phase selected for reverse phase HPLC should have low toxicity (e.g., for safety) and viscosity and be readily available in pure form. The mobile phase may be selected, e.g., water or water and an additional solvent selected from the group consisting of miscible lower alcohols (e.g. methanol, n-propanol, or isopropanol), tetrahydrofuran, dioxane, acetonitrile, and mixtures thereof.

In order to resolve the peak of 14-hydroxycodeinone and/or codeinone provided by the solutions utilized in the present invention, the temperature and pH of the system, among other factors, may be modified to preferred parameters, such as those discussed below. Preferably, the resolution between the 14-hydroxycodeinone and/or codeinone and any other component in the sample (e.g., oxycodone or hydrocodone) according to U.S.P. methodology is greater than or equal to 1.5, preferably at least 3, preferably at least 4, more preferably at least 5, and most preferably at least 6. Typically, the resolution between the particular pair of 14-hydroxycodeinone (or codeinone) and oxycodone is wider than the particular pair of 14-hydroxycodeinone (or codeinone) and hydrocodone.

In certain embodiments of the present invention, the mobile phase of the HPLC system is modified in order to control the pH on the column. In certain embodiments, the mobile phase is adjusted to a pH of from about 7.0 to about 11.0, to a pH of from about 7.0 to about 8.0, to a pH of from about 9.0 to about 10.0, to a pH of from about 9.3 to about 9.4, or to a pH of about 7.8. In certain embodiments, the ph is adjusted with a suitable agent such as, e.g., ammonium carbonate, ammonium hydroxide, phosphate buffer or acetic acid.

In certain embodiments, the signal:noise ratio of the system is preferably at least 5:1, preferably at least 10:1, more preferably at least 15:1 and most preferably at least 20:1.

The methods of the present invention include the use of at least one mobile phase, which acts as a carrier for the sample solution. The chemical interactions of the mobile phase and sample with the column determine the degree of migration and separation of the components of the sample. In certain preferred embodiments, the methods of the present invention include the use of a first mobile phase and a second mobile phase.

In certain embodiments, the methods of the present invention include performing HPLC through the use of isocratic elution (isocratic mobile phase) or gradient elution (gradient mobile phase). In isocratic elution, for example, compounds are eluted using a mobile phase having a constant composition. The compounds migrate through the column at onset, with each compound migrating at a different rate, resulting in separation of the compounds. In gradient elution, for example, different compounds may be eluted as the composition of the mobile phase changes, e.g., by increasing the strength and/or the concentration of the organic solvent. For example, the sample may be injected during application of a “weaker” mobile phase through the system. The mobile phase may be gradually or incrementally changed by, e.g., increasing the fraction of the mobile phase comprising the organic solvent, resulting in elution of retained compounds.

The mobile phase(s) for use in the HPLC methods of the present invention preferably includes an eluant comprising, for example and without limitation, acetonitrile, dioxane, ethanol, methanol, isopropanol, tetrahydrofuran, ammonium carbonate, water, or a mixture thereof. In one embodiment, the mobile phase comprises methanol. In another embodiment, the mobile phase comprises acetonitrile. In certain preferred embodiments, wherein the methods of the present invention utilize a first mobile phase and a second mobile phase, the first mobile phase may comprise one solvent or a combination of solvents, and the second mobile phase comprises one or more organic solvents.

Typical adsorbents in the HPLC column for use in the methods of the present invention include, for example and without limitation, IB-Sil C18, Prodigy ODS, Selectosil C18, Ultracarb ODS, Zorbax ODS, Kromasil C18, LiChrospher RP-18, Inertsil ODS-2, Nucleosil C18, Spherisorb ODS, Hypersil C18, Novapak C18, and Symmetry C18. Preferably, the adsorbent is Waters Symmetry C18. In certain embodiments, the column used is a difunctionally bonded, silica-based, reverse phase, octadecyl column (e.g., Water Atlantis® column).

In one embodiment of the present invention, the eluant for use in the HPLC methods of the present invention comprises phosphate buffer, methanol, acetonitrile, water and sodium dodecyl sulfate. In another embodiment, the eluant for use in the HPLC methods of the present invention comprises ammonium carbonate, methanol, water, acetic acid, ammonium hydroxide, phosphoric acid, or a mixture thereof.

In certain embodiments, the mobile phase(s) utilized in the present invention for isocratic elution comprises from about 50% (v/v) aqueous medium to about 85% aqueous medium, or from about 60% aqueous medium to about 75% aqueous medium. In a preferred embodiment, the aqueous medium comprises ammonium carbonate. In certain embodiments, the mobile phase comprises from about 50% methanol to about 15% methanol, or from about 40% methanol to about 25% methanol. In certain embodiments, the mobile phase comprises about 50% aqueous medium and about 50% methanol, preferably about 60% aqueous medium and about 40% methanol, or about 75% aqueous medium and about 25% methanol. In certain of the above ranges for methanol, acetonitrile can be substituted for all of, or a portion of, the methanol. Alternatively, an appropriate gradient elution profile may be selected by the skilled artisan to carry out the methods of the present invention.

In certain embodiments, the mobile phase(s) of the present invention is (are) delivered at a rate of from about 0.1 to about 2.0 mL per minute, preferably at a rate of about 0.7 mL per minute.

As used herein, an “HPLC compatible detector” is any detector capable of generating a measurable or detectable signal when a compound elutes from the column of an HPLC. Where component absorbance varies widely, it may be necessary to utilize more than one detector. A detector capable of detecting a desired component is not “incompatible” simply due to its inability to detect a non-desired component. The detector can be a refractive index detector, an ultraviolet detector, a fluorescent detector, a radiochemical detector, an electrochemical detector, a near-infrared detector, a mass spectrometric detector, a nuclear magnetic resonance detector, a light scattering detector, or any other detector known in the art.

In certain embodiments, the detector is an ultraviolet detector. In certain embodiments, the ultraviolet detector is selected from the group consisting of a fixed wavelength detector, a variable wavelength detector and a diode array detector. Preferably the ultraviolet detector is a fixed wavelength detector. In certain embodiments the fixed wavelength detector measures at a wavelength of from about 200 nm to about 275 nm, preferably at a wavelength of about 220 nm.

In certain embodiments, wherein the present invention comprises the use of two standards and one sample solution, the two standards preferably have a different concentration of 14-hydroxycodeinone or codeinone from each other. In certain embodiments, the 14-hydroxycodeinone or codeinone concentration of the first standard is from about 5 fold to about 500 fold greater, or from about 5 fold to about 100 fold greater, or from about 5 fold to about 50 fold greater, or about 10 fold greater than the 14-hydroxycodeinone or codeinone concentration of the second standard. In certain embodiments, the first standard comprises 14-hydroxycodeinone or codeinone in an amount of from about 50 ppm to about 500 ppm, or from about 75 ppm to about 200 ppm, or about 100 ppm; and the second standard includes 14-hydroxycodeinone or codeinone in an amount of from about 1 ppm to about 50 ppm, or from about 5 ppm to about 25 ppm, or about 10 ppm.

In certain embodiments utilizing one standard solution, the standard comprises 14-hydroxycodeinone in an amount of from about 1 ppm to about 500 ppm, or from about 1 ppm to about 20 ppm, or about 10 ppm.

In certain embodiments utilizing one standard solution, the standard comprises codeinone in an amount of from about 1 ppm to about 500 ppm, or from about 1 ppm to about 20 ppm, or about 10 ppm.

In certain embodiments, where the present invention comprises the use of two sample solutions wherein the concentrations of the samples are different, a first, more concentrated sample solution is used to detect the 14-hydroxycodeinone and a second, less concentrated sample solution is used to detect the oxycodone.

In certain embodiments of the present invention, the concentration of the sample of oxycodone for use in the first sample solution is from about 10 mg/mL to the solubility limit of the oxycodone; e.g., from about 50 mg/mL to about 150 mg/mL; or from about 15 mg/mL to about 35 mg/mL; about 50 mg/mL, or about 25 mg/mL.

In certain embodiments wherein the present invention utilizes no standard and two sample solutions and the concentrations of the samples are different, the concentration of oxycodone in the second sample solution is from about 2 times to about 500 times less than the concentration of oxycodone in the first sample solution; or from about 10 times to about 250 times less than the concentration of oxycodone in the first sample solution; or from about 50 times to about 100 times less than the concentration of oxycodone in the first sample solution. In a preferred embodiment, the concentration of oxycodone in the second sample solution is from about 0.01 mg/mL to about 10 mg/mL, or from about 0.10 mg/mL to about 2 mg/mL, or about 0.25 mg/mL.

In certain embodiments, the HPLC apparatus comprises an autoinjector with a preferable injection volume of from about 1 microliter to about 100 microliters, from about 10 microliters to about 100 microliters, from about 25 microliters to about 50 microliters, from about 1 microliter to about 10 microliters, about 25 microliters or about 5 microliters.

The following examples illustrate various aspects of the present invention. They are not to be construed to limit the claims in any manner whatsoever.

### EXAMPLE 1

In Example 1, 37.7 g of oxycodone HCl (35.4 g dry basis, ca. 500 ppm 14-hydroxycodeinone) was placed in a 500 mL Parr reaction bottle and combined with 0.55 g 5% Pd/C catalyst, 50% water wet (Johnson Matthey type 87L), and 182.2 g of 61.9% isopropanol/water (w/w). The mixture was placed under an inert atmosphere and heated with shaking to 45-50° C. Upon dissolution of all starting material, the pressure in the bottle was vented to the atmosphere and hydrogen pressure was applied (45 PSIG) for 4 hours. At the end of the hydrogenation, the hydrogen was vented off and the solution was allowed to cool to room temperature.

The next day, the mixture was heated to 75° C. to dissolve the crystallized solids and then suction filtered over a 0.2 μm PTFE membrane into a 1 L jacketed cylindrical flask (equipped with a condenser, a nitrogen atmosphere, a mechanical stirrer, a type K thermocouple, and a programmable refrigerated recirculator). The Parr bottle was rinsed with deionized water (11.7 g), which was added to the 1 L flask through the filter. Isopropanol (334.7 g) was added to the flask and the mixture was re-heated with stirring to 75° C. and held to dissolve any crystallized solids. The solution was cooled with stirring to 0-10° C. over 8 hours (linear ramp) and held at 0-10° C. for 20 hours. The crystallized solid was then collected by suction filtration and washed with 107 g of cold 95:5 isopropanol/water (w/w).

To remove isopropanol from product, the solvent-wet material was transferred to a drying dish and placed in a vacuum desiccator with an open container of deionized water. The solid was held in this manner, under vacuum, overnight. The material was then dried under vacuum at 60° C.

Analysis of the dried material using the low 14-hydroxycodeinone method of Examples 4 and 5 below gave a result of 6 ppm of 14-hydroxycodeinone.

Analysis of the dried material using the method of Example 7 below gave a result of <5 ppm of codeinone and 8 ppm of 14-hydroxycodeinone.

### EXAMPLE 2

In Example 2, 35.0 g of oxycodone HCl (33.3 g dry basis, ca. 4000 ppm 14-hydroxycodeinone) was placed in a 500 mL Parr reaction bottle and combined with 0.49 g 5% Pd/C catalyst, 50% water wet (Johnson Matthey type 87L), and 159.9 g of 62.3% isopropanol/water. The mixture was placed under an inert atmosphere and then heated with shaking to 45-50° C. Upon dissolution of the starting material, the pressure in the bottle was vented to the atmosphere and hydrogen pressure was applied (45 PSIG). After 5.25 hours of shaking, the hydrogen was vented off, and the solution was allowed to cool to room temperature. The mixture was re-heated the next day and hydrogenation was continued for 4.75 hours.

The mixture was heated to 75° C. and then suction filtered over a 0.2 μm PTFE membrane into a 1 L jacketed cylindrical flask (equipped with a distillation head, a nitrogen atmosphere, a mechanical stirrer, a type K thermocouple, and a programmable refrigerated recirculator). The Parr bottle was rinsed with deionized water (11.7 g), which was added to the 1 L flask through the filter.

Isopropanol (295.6 g) was added to the flask and the mixture was heated to boiling (ca. 81° C.). To remove water and increase the yield, isopropanol/water azeotrope was distilled from the flask until 305.7 g had been collected. Fresh isopropanol (305.6 g) was added and the distillation head was removed and replaced with a condenser.

The mixture was cooled with stirring from boiling to 0-10° C. over 8 hours (linear ramp) and held at 0-10° C. for 20 hours. The crystallized solid was then collected by suction filtration and washed with 107 g of cold 95:5 isopropanol/water. The material was dried as described in Example 1.

Analysis of the dried material using the low 14-hydroxycodeinone method of Examples 4 and 5 below gave a result of <5 ppm of 14-hydroxycodeinone.

Analysis of the dried material using the method of Example 7 below gave a result of <5 ppm of codeinone and <5 ppm of 14-hydroxycodeinone.

### EXAMPLE 3

In Example 3, 27.83 g of oxycodone free-base, water wet (24.57 g dry basis, 0.0779 mol, ca. 3000 ppm 14-hydroxycodeinone), 39.8 g of deionized water, 81.9 g of isopropanol, 0.49 g 5% Pd/C catalyst, 50% water wet (Johnson Matthey type 87L), and conc. HCl (11.3 g, 0.117 mol, 1.50 equivalents based on 37.7% HCl assay) were combined in a 500 ml Parr shaker bottle.

The mixture was placed under an inert atmosphere and heated to 75° C. with shaking. The pressure in the bottle was relieved, and the system was pressurized with hydrogen (45 PSIG). The solution was held under these conditions for 21.7 hours. Analysis by HPLC showed that the ratio of the area of the 8,14-dihydroxy-7,8-dihydrocodeinone peak to that of oxycodone was reduced from 0.29% to 0.04% during this time.

The hydrogen pressure was vented and the system was placed under an inert atmosphere. In order to prevent further dehydration of any residual 8,14-dihydroxy-7,8-dihydrocodeinone, the pH of the solution was adjusted from 0.5 to 1.8 with 20.7 g NaOH saturated isopropanol (some solid sodium hydroxide was also present).

The solution was re-heated to 75° C. and then pressure filtered through a 0.2 μm PTFE membrane filter housed in heat-traced 47 mm SS filter holder into a 500 ml jacketed cylindrical reactor (condenser, N₂, mechanical stirrer, programmable refrigerated recirculator). The Parr bottle was rinsed with 8.6 g of deionized water, which was added to the flask through the filter.

Isopropanol (222.5 g) was added to the solution in the flask and the resulting slurry was heated to approximately 75° C. to re-dissolve the solids. After reaching the desired temperature, the solution was held for two hours (to simulate typical processing times). No 14-iydroxycodeinone was detected in a sample of the crystallization mixture after this hold.

The circulator was set to cool from 80° C. to 0° C. over 8 hours. Approximately 24 hours after starting the cooling program, the solids were collected by suction filtration and washed three times with 95:5 isopropanol/water (232.8 g total). The material was dried as described in Example 1.

Analysis of the dried material using the low 14-hydroxycodeinone method of Examples 4 and 5 below gave a result of 5 ppm of 14-hydroxycodeinone.

Analysis of the dried material using the method of Example 7 below gave a result of <5 ppm of codeinone and 10 ppm of 14-hydroxycodeinone.

### EXAMPLE 4

### Analysis of Sample to Determine 14-Hydroxycodeinone Level

The products of Examples 1-3 were analyzed to determine the level of 14-hydroxycodeinone under 100 parts per million (PPM) level by a HPLC method using a Waters Atlantis 5 μm dC18, 3×250 mm column maintained at 50° C. and isocratic elution using pH 9.35, 17 mM ammonium carbonate buffer and methanol (60:40). Quantitation was achieved by measuring the peak area response with UV detection at 220 nm using external standard. This method utilized mobile phase with volatile components that are compatible with LC/MS analysis.

The reagents used were as follows:

1. Ammonium carbonate, analytical reagent grade (Aldrich);

2. Water, HPLC grade;

3. Methanol, HPLC grade;

4. Acetic acid, reagent grade (J. T Baker Glacial Acetic Acid);

5. Ammonium hydroxide, reagent grade;

6. Phosphoric acid, about 85%, A.C.S. reagent;

7. 14-Hydroxycodeinone reference material from Albany Molecular Research, Inc.

The equipment used was as follows:

A. HPLC System 1. HPLC system capable of delivering 0.4 mL/minute of mobile phase (Waters Alliance);

2. UV/Visible detector set to monitor the eluant at 220 nm (Waters 2487 UV/Vis);

3. Autosampler capable of injecting 6 μL;

4. Integrator or suitable data recording system (Waters Millennium32 chromatograph system.);

5. Waters, Atlantis dC18 column, 3×250 mm, 5 μm;

6. Column heater capable of maintaining a constant temperature of 50° C.;

7. On-line vacuum degasser.

B. Equipment for Mobile Phase Preparation

1. pH meter, preferably with automatic temperature compensation (ATC);

2. Ultrasonic bath, Model 5200, Branson;

3. 0.45-μm membrane filters for aqueous solvent, Whatman or Millipore, Cellulose acetate or Nylon.

### Solutions

A. 17 mM Ammonium Carbonate, pH 9.35

1.6±0.1 g of ammonium carbonate was weighed and placed into a 1-L beaker. 1000 mL of water was added to the beaker and stirred with a magnetic stirrer until the ammonium carbonate was dissolved. The pH was adjusted to 9.35-9.40 with ammonium hydroxide.

B. Mobile Phase

400 mL of HPLC-grade methanol was mixed with 600 mL of 17 mM ammonium carbonate, pH 9.35-9.40 prepared above. The mixture was filtered through solvent membrane filters and then degassed using an on-line vacuum degasser in the HPLC system.

C. 0.85% Phosphoric Acid Solution

10.0 mL of 85% H₃PO₄ was pipetted into a 1 liter volumetric flask and diluted to volume with water and mixed thoroughly.

D. 14-Hydroxycodeinone Working Reference Standard Solutions

A stock 14-hydroxycodeinone standard solution was prepared by weighing 25±2 mg of 14-hydroxycodeinone reference material and transferring it into a 250-mL volumetric flask. Approximately 100 mL of 0.85% H₃PO₄ solution was added to the flask and sonicated for approximately 2 minutes or until dissolved. The solution was diluted to volume with 0.85% H₃PO₄ solution and mixed thoroughly. This was the stock 14-hydroxycodeinone standard solution.

A working solution of 100 ppm 14-hydroxycodeinone standard solution for system suitability was prepared by pipetting 5.0 mL of the stock 14-hydroxycodeinone standard solution into a 100-mL volumetric flask, diluting the solution to volume with water and mixing thoroughly.

A working solution of 10 ppm 14-hydroxycodeinone standard solution for sensitivity was prepared by pipetting 5.0 mL of working 100 ppm 14-hydroxycodeinone standard solution into a 50-mL volumetric flask, diluting the solution to volume with water and mixing thoroughly.

E. Hydrocodone Working Reference Standard Solution

Stock Hydrocodone Standard Solution was prepared by weighing 25±2 mg of Hydrocodone reference material and transferring contents into a 250-mL volumetric flask. Approximately 100 mL of 0.85% H3PO4 solution was added to the flask and sonicated for approximately 2 minute or until dissolved. The solution was diluted to volume with 0.85% H3PO4 Solution and mixed thoroughly.

F. Sample Solutions

A sample solution was prepared by weighing about 250 mg oxycodone API sample into a scintillation vial. 5.0 mL of water was pipetted into the vial to dissolve the sample. The vial was tightly capped and sonicated for approximately 5 minutes or until the sample was dissolved. The contents were then shaken and mixed thoroughly.

G. Resolution Test Mixture (RTM) Solution

A solution containing two components, 14-hydroxycodeinone and hydrocodone, was prepared from the respective stock standard solutions.

The Resolution Test Mixture (RTM) was prepared by pipetting separately 10.0 mL of each stock standard solution of hydrocodone above and 14-hydroxycodeinone above into the same 100 mL volumetric flask and diluted to volume with a sufficient amount of water and mixed thoroughly.

H. HPLC Conditions

The HPLC conditions were as follows:

Column: Waters, Atlantis dC18, 3×250 mm, 5 μm.

Column temperature: 50° C.

Detector wavelength: 220 nm

Injection volume: 6 μl

Quantitation: Peak area of 14-hydroxycodeinone

Mobile Phase: (60:40) 17 mM ammonium carbonate, pH 9.35-9.40: Methanol

Flow low rate: 0.4 mL/minute

Run time: 70 minutes for the samples and 40 minutes for the standard and RTM solutions

I. Resolution Test Mixture (RTM) Test

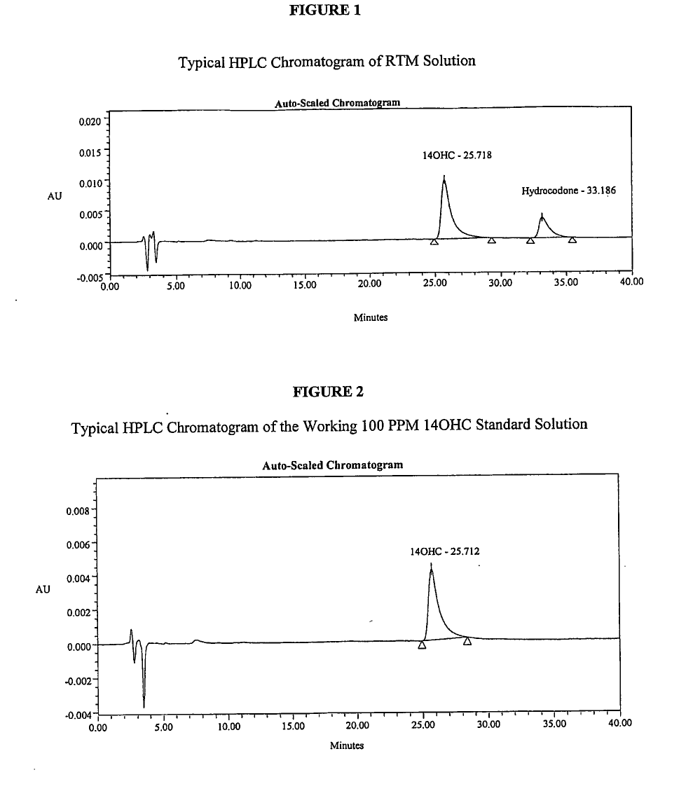

Before performing the system suitability test, a new column was equilibrated over night (at least 12 hours) by pumping mobile phase through it at 0.4 mL/min. After the new column was equilibrated, 6 μL of RTM solution was injected into the equilibrated system to ensure that the two eluted component peaks did not interfere with one another. A typical separation of the system suitability testing solution is shown in FIG. 1.

J. System Suitability Test

A system suitability test was performed by injecting the Working 100 ppm 14-hydroxycodeinone standard solution into the system and by performing the system suitability test as described in the USP <621> by making six different runs of 6 μL injections. The system suitability test results met the following criteria listed in Table 1 below.

TABLE 1

| Test No. | System Suitability Test | Specification |
|---|---|---|
| 1 | RSD of peak areas for 14- | RSD ≦3.0% |
| | hydroxycodeinone (1) | |
| 2 | RSD of retention time for | RSD ≦2.0% |
| | 14-hydroxycodeinone (1) | |
| 3 | Column Efficiency | N ≧2000 |
| | (Theoretical Plates of 14- | |
| | hydroxycodeinone) (1) | |
| 4 | Resolution between 14- | R ≧1.5 |
| | hydroxycodeinone and | |
| | Hydrocodone (2) | |
| 5 | Signal to noise ratio (3) | S/N ≧10 |

Note:
(1) the working 100 ppm 14-hydroxycodeinone standard solution for Test Nos. 1 to 3 was used.
(2) the RTM for Test No. 4 was used.
(3) the working 10 ppm 14-hydroxycodeinone standard solution for Test No. 5 was used.

Before starting the experiment, 6 μL of water was injected to ensure that there were no interfering peaks co-eluting with the peak for 14-hydroxycodeinone. The following procedure was then conducted.

The working 100 ppm 14-hydroxycodeinone standard solution was injected six times in different runs, and the system was checked to verify that it met the system suitability test specifications as listed for Test Nos. 1, 2 and 3 in Table 1 above.

The RTM solution was injected and run once in the HPLC system to confirm that the system met the system suitability test specification as listed for Test No. 4 in Table 1 above.

The working 10 ppm 14-hydroxycodeinone standard solution was injected and run once in the HPLC system to confirm that the system had signal-to-noise ratio S/N greater than or equal to 10, as listed in the specification for Test No. 5 in Table 1 above.

After the system passed all of the above tests, the following HPLC procedure was performed.

The working 100 ppm 14-hydroxycodeinone standard solution and the working 10 ppm 14-hydroxycodeinone standard solution were each injected separately. Both working standard solutions were used to quantitate the samples. The setting and integration parameters are listed in Table 2 below.

TABLE 2

| Integration Setting | Parameters |
|---|---|
| Minimum area | 0 |
| Minimum height | 0 |
| Threshold | 2 |
| Peak width | 90.00 |
| Inhibit integration: 0.01 to 20 minutes | Eliminates solvent front |

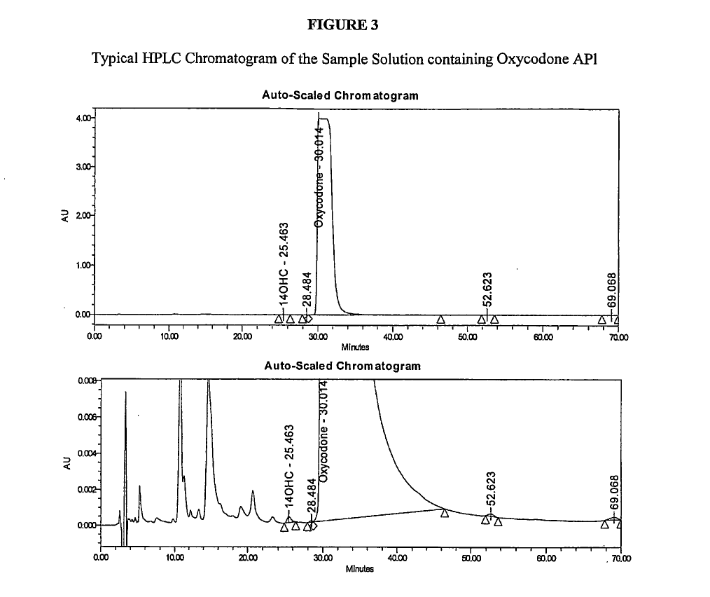

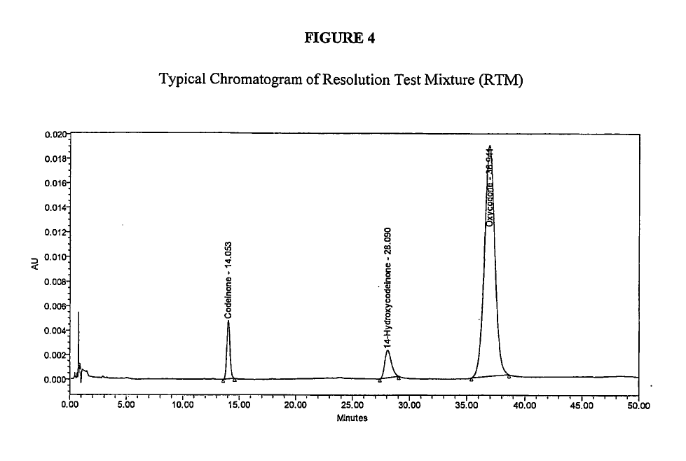

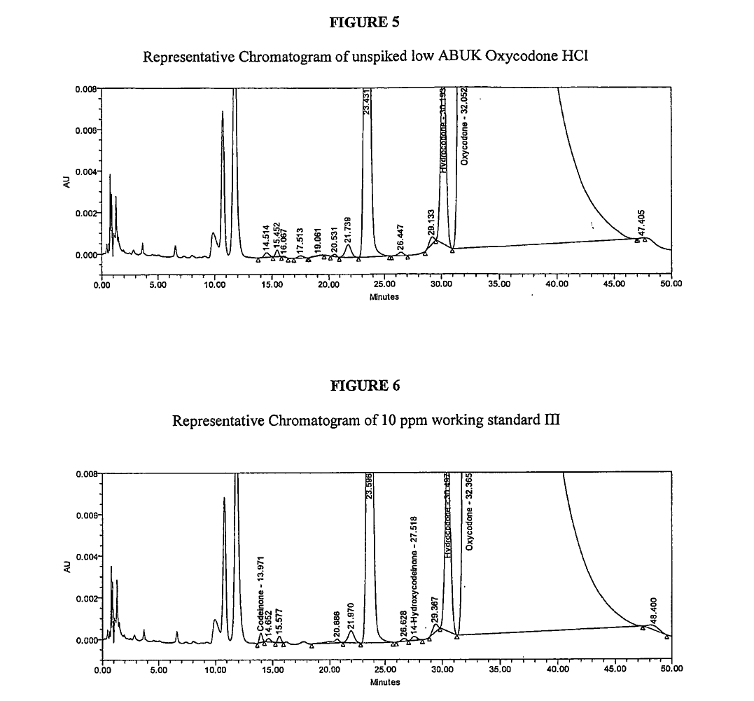

Typical HPLC chromatograms for the working 100 ppm 14-hydroxycodeinone standard solution and the oxycodone API sample solution are shown in FIG. 2 and FIG. 3 respectively. Retention times of the 14-hydroxycodeinone and other related substances are presented in Table 3 below.

TABLE 3

| | Relative Retention Time vs. |
|---|---|
| Peak ID | Oxycodone (RRT) |
| Oxycodone-N-Oxide (ONO) | 0.16 |
| Noroxycodone | 0.31 |
| Oxymorphone | 0.45 |
| 7,8-Dihydro-8,14-Dihydroxycodeinone | 0.58 |
| (DDC) | |
| 14-Hydroxycodeine | 0.73 |
| 14-Hydroxycodeinone | 0.79 |
| 6-α-Oxycodol | 0.96 |
| Hydrocodone | 0.95 |
| Oxycodone | 1.0 |
| Thebaine | 1.89 |

The following calculations were performed using the results obtained above.

Using Millennium®, software, the parameters were entered as follows:

In the sample set, the standard concentrations for both working standards (10 and 100 ppm) were calculated as follows:

$$100 PPM std . conc . = \frac{{W}_{std} corrected for purity}{250} \times 0.05$$

$$10 PPM std . conc . = \frac{{W}_{std} corrected for purity}{250} \times 0.005$$

where W_{std} is the weight of standard.

The following were also entered:

Sample weight=weight of sample in mg

Dilution=5 ml (sample dilution)

Label claim=0.0001 (to convert the results in PPM.

The amount of 14-hydroxycodeinoneOHC in oxycodone sample in ppm can be determined automatically from a linear calibration curve using the two standards (100 PPM and 10 PPM) and the equation used in the calculation below.

$$PPM of 14 OHC = \frac{{A}_{sam} - {Y}_{intercept}}{Slope} X \frac{D}{{W}_{sam}} \times 1000000$$

where:

Aₛₐₘ=peak area of 14OHC

Y_{intercept}=Y intercept from a linear regression line using the two standards

Slope=slope from a linear regression line using the two standards

D=5.0 (sample dilution factor)

Wₛₐₘ=sample weight in mg

1000000=Convention factor to convert the result to PPM

### EXAMPLE 5

3.0 g of oxycodone hydrochloric salt 154 ppm 14-hydroxycodeinone was dissolved in 20 mL water to afford a clear solution in a 250 mL Parr reaction bottle. To the solution, 0.05 g 5% Pd/C catalyst, 50% water wet (Johnson Matthey type 87L) and 1 mL formic acid 88% were added. The mixture was placed under inert atmosphere without hydrogen feed and then heated to 45° C.-50° C. After 2 hours of shaking, a sample was taken to check the disappearance of 14-hydroxycodeinone. The sample showed no 14-hydroxycodeinone by the HPLC method described in Examples 4 and 5 above

The solution was then suction filtered over a 0.2 micron PTFE membrane to remove the catalyst. An aliquot of 2 mL was taken out of about 18 mL filtrate solution. To this solution, 2.0 mL isopropyl alcohol was added to obtain a clear solution, followed by 4.0 mL of ethyl acetate. The solution was stirred, cooled and kept at 0-5° C. for 20 hours to afford oxycodone hydrochloride crystals. The crystalline solid was isolated by suction filtration. The wet solid was dried in an oven at 50° C. and 10 mmHg pressure. The dried solid weighed 0.12 g.

Analysis using the HPLC method in Example 4 above indicated that about 11 ppm 14-hydroxycodeinone were present in the oxycodone hydrochloride salt composition. In another aliquot of 2 mL of the filtrate solution, 16-18 mL of isopropyl alcohol was added to the concentrated oxycodone hydrochloride solution followed by crystallization and drying. The procedure afforded oxycodone hydrochloride salt containing about 6.8 ppm 14-hydroxycodeinone.

### EXAMPLE 6

### Analysis of Sample to Determine 14-Hydroxycodeinone and Codeinone

The products of Examples 1-3 were analyzed by the following method to determine the amount of codeinone and 14-hydroxycodeinone present. This method uses a Waters Symmetry C₁₈ column maintained at 40° C. with isocratic elution using a mobile phase of sodium phosphate buffer, sodium dodecyl sulfate (SDS), acetonitrile (ACN), and methanol (MeOH).

The reagents used were as follows:

1. Water, HPLC grade or equivalent;

2. Phosphoric acid, 85%, HPLC reagent grade or equivalent;

3. Sodium phosphate monobasic, monohydrate, Enzyme grade or equivalent;

4. Sodium dodecyl sulfate (99%+), Ultrapure, Fluka or equivalent;

5. Acetonitrile, HPLC grade or equivalent;

6. Methanol, HPLC grade or equivalent;

7. Sodium hydroxide, ACS reagent grade or equivalent;

8. Oxycodone HCl with low ABUK to be used as part of the matrix in standard preparation;

9. Codeinone reference material from Rhodes Technologies or equivalent;

10. 14-Hydroxycodeinone reference material from Albany Molecular Research or equivalent

The equipment used was as follows:

A. HPLC System

For this analysis, an HPLC system with a dual wavelength detector was used that was able to operate under isocratic conditions at a flow rate of 0.7 mL per minute with UV detection @ 220 nm, and a column temperature of 40° C.

B. Mobile Phase Filtration System

For this analysis, an HPLC vacuum filtration apparatus with a nylon membrane filter (0.45 μm) was used.

Solutions

A. 50% Sodium Hydroxide Solution (W/V)

50 g of sodium hydroxide pellets were weighed and transferred into a 100-mL volumetric flask. 60-mL of water was then added and sonicated until the pellets were completely dissolved. The pellets were diluted to volume with water and mixed well. (Commercially available 50% w/v NaOH solution may also be used.)

B. Phosphoric Acid Solution I (˜8.5% H₃PO₄)

10 ml of concentrated phosphoric acid (85%) was transferred into a 100 ml volumetric flask containing approximately 50 ml of water. The volume was diluted with water and then mixed.

C. Phosphoric Acid Solution II (˜0.85% H3PO4)

10-mL of 85% phosphoric acid was pipetted into a 1000-mL volumetric flask, diluted to volume with water and mixed well. This was the diluent for the sample and standard preparation.

D. Mobile Phase

3.45 g±0.1 g of sodium phosphate monobasic monohydrate was weighed into a 1-L flask. 1000 mL of water was added and then stirred with a magnetic stirrer until dissolved. 5.41 g±0.1 g of sodium dodecyl sulfate was added and mixed well until dissolved. This solution was filtered using vacuum filtration with a 0.45-μm nylon membrane filter. The pH of this solution was adjusted with 50% NaOH solution to a final pH of 7.50±0.05.

722.5 ml of the above solution was then mixed with 157.5 mL of acetonitrile, then 120 mL of methanol was added to the solutions and mixed well. The final pH was adjusted to 7.80±0.01 with ˜8.5% phosphoric acid solution. The mobile phase was sonicated for about 5 minutes to remove dissolved air.

Standard Solution Preparation Calculated Relative to Dried Samples

A. Codeinone/14-Hydroxycodeinone Stock Solution I

25±1 mg of both codeinone and 14-hydroxycodeinone reference materials were weighed and transferred into a 100-mL volumetric flask, diluted to volume and dissolved with ˜0.85% phosphoric acid solution II.

B. 100 PPM Stock Standard II

1-ml of stock solution I was pipetted into a 50-ml volumetric flask, diluted to volume with ˜0.85% phosphoric acid solution II and then mixed.

C. 10 PPM Working Standard III

500±5 mg of Oxycodone low ABUK material was weighed into a 10-ml volumetric flask. 1-ml of stock standard II was pipetted and diluted to volume with ˜0.85% phosphoric acid solution II and mixed.

D. Unspiked Oxycodone Solution

500±5 mg of Oxycodone low ABUK material was weighed into a 10-ml volumetric flask, diluted to volume with ˜0.85% phosphoric acid solution II and mixed. (This solution was used to calculate the residual content of both Codeinone and 14-Hydroxycodeinone in the working standard).

E. Resolution Test Mixture (RTM)

1.0-ml of the Codeinone/14-Hydroxycodeinone stock solution I was pipetted into a 50-ml volumetric flask. Using a micropipette, 100 μl of the unspiked Oxycodone solution was transferred and diluted to volume with ˜0.85% phosphoric acid solution II. The concentration of Codeinone, 14-Hydroxycodeinone, and Oxycodone was approximately 100 ppm.

Sample Preparations

A. 50 mg/mL Oxycodone HCl Sample Solution

500±5 mg of Oxycodone HCl was weighed, in duplicate, into separate 10-mL volumetric flasks for each of Examples 1, 2 and 3. The Oxycodone HCl was then diluted to volume with the ˜0.85% phosphoric acid solution II and swirled to dissolve the sample. A sufficient amount of this sample was transferred to an HPLC vial for injection.

HPLC Conditions

The HPLC conditions were set as follows:

TABLE 4
HPLC Conditions

| Parameter | Condition |
|---|---|
| HPLC Column | Symmetry C₁₈, 3.0 × 150 mm, 3.5 μm particle size |
| Mobile Phase | 18 mM phosphate/13 mM SDS pH = 7.50: ACN: |
| | MeOH (72.25:15.75:12.0) pH = 7.80 ± 0.01 |
| Flow Rate* | 0.7 mL/min |
| Column | 40° C. |
| Temperature | |
| Detection | 220 nm |
| Injection Volume | 5 μL |
| Run Time | 50 minutes |

*Parameter may be adjusted to achieve retention times.

System Suitability

One injection (5-μL) of a blank solution (˜0.85% phosphoric acid solution II) was made, followed by one injection of the RTM to determine if there was any interfering peaks in the blank solution. 6 injections of the working standard III were made. The system suitability injections were then tested to verify that they met the system suitability criteria as shown in Table 2.

TABLE 5
System Suitability Criteria

| | Acceptance |
|---|---|
| Parameter | Criteria |
| Resolution between Codeinone and 14-Hydroxycodeinone | NLT 8 |
| Resolution between 14-Hydroxycodeinone and Oxycodone | NLT 2 |
| Tailing factor for Oxycodone | 0.7-2.0 |
| Relative retention times for Codeinone based on Oxycodone | Approx. 0.44 |
| Relative retention times for 14-Hydroxycodeinone based on | Approx. 0.85 |
| Oxycodone | |
| % RSD of 6 system suitability injections for Codeinone and | NMT 20% |
| 14-Hydroxycodeinone | |

The expected retention times were as follows:

| Components | Expected Retention Times |
|---|---|
| Codeinone | 14 ± 2 min |
| 14-Hydroxycodeinone | 27 ± 4 min |
| Oxycodone | 32 ± 6 min |

Injection Procedure

Once the column was equilibrated, the sample and standard solutions were injected according to the following sequence of Table 3:

TABLE 6

| | |
|---|---|
| Blank (diluent) | 1 injection |
| Resolution solution | 1 injection |
| Working Standard III | 6 injections for RSD, last 2 |
| | injections for calibration |
| Blank (diluent) | 2 injection |
| Unspiked Oxycodone solution | 2 injection |
| Sample 1 Prep# 1 | 2 injections |
| Working Standard III | 2 injections |
| Sample 1 Prep# 2 | 2 injections |
| Sample 2 Prep# 1 | 2 injections |
| Sample 2 Prep# 2 | 2 injections |
| Working Standard III | 2 injections |
| Sample 3, Prep# 1 | 2 injections |
| Sample 3, Prep# 2 | 2 injections |
| Working Standard III | 2 injections |

The Codeinone and 14-Hydroxycodeinone peaks were identified using the relative retention times as discussed above.

Calculations

The responses of Codeinone and 14-Hydroxycodeinone peaks were measured and recorded. The content of Codeinone and 14-Hydroxycodeinone was calculated in ppm using the following equation:

$$\begin{matrix}ppm = \frac{Rs \times Wstd}{Rstd \times Ws} \times \frac{1}{100} \times \frac{1}{50} \times \frac{1}{10} \times \frac{10}{1} \times \frac{1 \text{,} 000 \text{,} 000}{1} \\ = \frac{Rs \times Wstd \times 200}{Rstd \times Ws}\end{matrix}$$

Where:
ppm=Parts per millions of codeinone or 14-Hydroxycodeinone in Oxycodone HCl
Rs=Response of Codeinone or 14-Hydroxycodeinone in Sample Solution.
Rstd=Response of Codeinone or 14-Hydroxycodeinone in Standard Solution minus the response of unspiked standard
Wstd=Weight of Standard, corrected for purity, mg
Ws=Weight of Sample, mg
1000000=Conversion Factor for ppm % Cod/14-HOCD=ppm/10,000

The results for Examples 1 gave a result of <5 ppm of codeinone and 8 ppm of 14-hydroxycodeinone.

The results for Examples 2 gave a result of <5 ppm of codeinone and <5 ppm of 14-hydroxycodeinone.

The results for Examples 3 gave a result of <5 ppm of codeinone and 10 ppm of 14-hydroxycodeinone.

Many other variations of the present invention will be apparent to those skilled in the art and are meant to be within the scope of the claims appended hereto.

1. A method of determining the amount of 14-hydroxycodeinone contained in an oxycodone preparation comprising:
(a) preparing for analysis by a detection system a standard solution comprising 14-hydroxycodeinone in a known concentration;
(b) preparing for analysis by the detection system a sample solution comprising oxycodone from the oxycodone preparation;
(c) analyzing the standard solution using the detection system to obtain a measurable quantification of 14-hydroxycodeinone in the known concentration;
(d) analyzing the sample solution of step (b) using the detection system to obtain a measurable quantification of 14-hydroxycodeinone in the sample solution; and
(e) determining the amount of 14-hydroxycodeinone in the oxycodone preparation based on a comparison of the quantifications obtained for the standard solution with the quantification obtained for the sample solution.

2. A method of determining the amount of 14-hydroxycodeinone contained in an oxycodone preparation comprising:
(a) preparing for analysis by a detection system a standard solution comprising 14-hydroxycodeinone in a known concentration;
(b) preparing for analysis by the detection system a sample solution of oxycodone from the oxycodone preparation, wherein the concentration of oxycodone in the sample solution is from about 10 mg/mL to the solubility limit of the oxycodone;
(c) analyzing the standard solution using the detection system to obtain a measurable quantification of 14-hydroxycodeinone in the known concentration;
(d) analyzing the sample solution of step (b) using the detection system to obtain a measurable quantification of 14-hydroxycodeinone in the sample solution; and
(e) determining the amount of 14-hydroxycodeinone in the oxycodone preparation based on a comparison of the quantifications obtained for the standard solution with the quantification obtained for the sample solution.

3. The method of claim 1, wherein the detection system is an HPLC system and the quantification is a measurable peak area.

4. The method of claim 2, wherein the detection system is an HPLC system and the quantification is a measurable peak area.

5. The method of claim 1, wherein the concentration of the standard solution is about 10 ppm.

6. The method of claim 1, wherein the concentration of oxycodone in the sample solution is about 50 mg/mL.

7. The method of claim 3, wherein the standard solution, the sample solution, or both are adjusted to a pH of about 7.0 to about 11.0.

8. The method of claim 3, wherein the HPLC system of the present invention has a column maintained at a temperature of from ambient temperature to about 60 degrees C., preferably at about 40 degrees C., to obtain a measurable peak area of 14-hydroxycodeinone.

9. The method of claim 1, which can provide for the detection of 14-hydroxycodeinone from about 5 ppm to about 100 ppm.

10. The method of claim 1, which can provide for the detection of 14-hydroxycodeinone from about 5 ppm to about 50 ppm.

11. The method of claim 1, which can provide for the detection of 14-hydroxycodeinone from about 5 ppm to about 25 ppm.

12. The method of claim 1, which can provide for the detection of 14-hydroxycodeinone from about 5 ppm to about 10 ppm.

13. The method of claim 1, which can provide for the detection of 14-hydroxycodeinone at about 5 ppm.

14. The method of claim 1, which can provide for the detection of 14-hydroxycodeinone at an amount less than about 5 ppm.

15. The method of claim 3, wherein the HPLC system comprises a mobile phase adjusted to a pH of about 7.0 to about 11.0, or about 7.0 to about 8.0.

16. The method of claim 15, wherein the mobile phase is adjusted to a pH of about 7.8.

17. The method of claim 3, wherein the HPLC system is selected from the group consisting of an adsorption chromatography system, an ion-exchange chromatography system and a size exclusion chromatography system.

18. The method of claim 17, wherein the HPLC system is an adsorption chromatography system.

19-50. (canceled)

51. A method of determining the amount of codeinone contained in an oxycodone preparation comprising:
(a) preparing for analysis by a detection system a standard solution comprising codeinone in a known concentration;
(b) preparing for analysis by the detection system a sample solution comprising oxycodone from the oxycodone preparation;
(c) analyzing the standard solution using the detection system to obtain a measurable quantification of codeinone in the known concentration;
(d) analyzing the sample solution of step (b) using the detection system to obtain a measurable quantification of codeinone in the sample solution; and
(e) determining the amount of codeinone in the oxycodone preparation based on a comparison of the quantifications obtained for the standard solution with the quantification obtained for the sample solution.

52. A method of determining the amount of codeinone contained in an oxycodone preparation comprising:
(a) preparing for analysis by a detection system a standard solution comprising codeinone in a known concentration;
(b) preparing for analysis by the detection system a sample solution of oxycodone from the oxycodone preparation, wherein the concentration of oxycodone in the sample solution is from about 10 mg/mL to the solubility limit of the oxycodone;
(c) analyzing the standard solution using the detection system to obtain a measurable quantification of codeinone in the known concentration;
(d) analyzing the sample solution of step (b) using the detection system to obtain a measurable quantification of codeinone in the sample solution; and
(g) determining the amount of codeinone in the oxycodone preparation based on a comparison of the quantifications obtained for the standard solution with the quantification obtained for the sample solution.

53-100. (canceled)
